(19) United States
(12) Patent Application Publication
(10) Pub. No.: US 2013/0177614 A1
(43) Pub. Date: Jul. 11, 2013

(54) METHOD OF USE OF STABILIZED NON-PLANT-DERIVED GROWTH FACTOR IN SKIN CARE

(71) Applicants: Einar Mantyla, Reykjavik (IS); Bjorn Larus Orvar, Kopavogur (IS); Hakon Orn Birgisson, Reykjavik (IS)

(72) Inventors: Einar Mantyla, Reykjavik (IS); Bjorn Larus Orvar, Kopavogur (IS); Hakon Orn Birgisson, Reykjavik (IS)

(21) Appl. No.: 13/542,889

(22) Filed: Jul. 6, 2012

(30) Foreign Application Priority Data
Jul. 6, 2011 (IS) 8967

(51) Int. Cl. A61K 38/18; A61K 8/64; A61Q 19/00; A61K 31/728

(52) U.S. Cl. CPC A61K 38/1866; A61K 31/728; A61K 38/1808; A61K 38/1841; A61K 8/64; A61Q 19/00
USPC 424/401; 514/7.6; 424/450; 530/399; 514/8.1; 514/9.6; 514/8.9

(57) ABSTRACT

Cosmetic and dermatologic compositions for skin care, containing a non-plant derived growth factor, purified from a host organisms or host cells such as bacterial, yeast, or animal cells, including insect cells, or a mixture of growth factors in purified form, for use in topical therapeutics, dermatology and cosmetics. Importantly this invention provides stabilized, safer growth factors available for use for cosmetic and topical treatment. Preferred composition comprises a non-plant derived growth factor and hyaluronic acid. The skin-care/dermatological compositions with stabilized growth factor do not carry the risk of unwanted breakdown products and the resulting loss of activity of the composition.

### FIELD OF THE INVENTION

The present invention generally relates to cosmetic and dermatological compositions comprising stabilized growth factors and cytokines for skin care, dermatological applications and methods for making skin care products. In particular, this invention relates to stabilized heterologous growth factors and their use in cosmetic and pharmaceutical products.

### BACKGROUND OF THE INVENTION

Skin is the biggest organ of the human body carrying out various functions such as protection, barrier, temperature controlling, excretion and respiration. With time and ageing, those functions rapidly decline, and a variety of physiological changes occur to the skin. These changes are manifested in the decrease in the thickness of epidermis, dermis and subcutaneous tissue, which are the main components of skin. Changes in lipid composition undermine the moisture barrier role of lipid layers and resulting in the dryness of skin. Further, with age, the occurrence of age spots, freckles, pigmentation or various skin lesions also increases.

An age-dependent decrease in epidermal turnover rate is involved in accumulation of low quality Stratum Corneum, resulting in senile xerosis, undue pigmentation and fine wrinkles. This may, in part, be due to aberrant keratinocyte differentiation.

Environmental components such as pollution and UV-rays, can speed up the ageing of the skin. Reactive oxygen species and free radicals and some physiological states such as fatigue or stress are particularly detrimental to proteins, nucleic acids and membrane lipids, leading to the aging of the skin. Accordingly, there have been many studies on the occurrence of the wrinkles, age spots or freckles, the loss of skin elasticity, the pigmentation, and the dryness and cracking of skin.

A variety of cosmetic compositions have been developed in order to prevent or slow down the problems of aging of the skin and skin wrinkles with the aim of improving wrinkles, sagging and the reduction in elasticity of skin caused by sunlight. Japanese Patent Laid-open Publication No. Hei 5-246838 discloses a method for improving wrinkles of skin by the synthesis of collagen. It teaches that the activity of collagenase that decomposes collagen to promote collagen metabolism might be reduced with aging, leading to the increase of cross-link collagens and the increase of skin wrinkles.

### Skin Conditions:

### Psoriasis:

Psoriasis is a skin condition resulting in red coloration of the skin and scaly patches and peeling of skin at the spot. Psoriatic patches are sites of excess skin production due to changes in cell development and rate of cell division in the epidermis resulting from altered growth behavior of these cells. It is known that cells at the site of psoriatic patch produce cell signaling compounds resulting in inflammatory response. It has been hypothesized that psoriasis is a form of immune response that may result in self-incompatibility that can be triggered by external conditions such as infections.

### Eczema:

Eczema is a form of dermatitis, or inflammation of the epidermis. The term eczema is broadly applied to a range of persistent skin conditions. These include dryness and recurring rashes that can have one or more of the symptoms redness, skin edema (swelling), itching and dryness, crusting, flaking, blistering, cracking, or even oozing or bleeding. Dermatitis is often treated with corticosteroids. Because of the risks associated with corticoids such as thinning of skin, steroids must be sparingly applied only to control an episode of eczema. Eczema is classified into several forms comprising; Seborrhoic eczema, atopic eczema, xerotic eczema, dyshidrosis, discoid eczema,venous eczema, dermatitis herpetiformis, neurodermatitis, autoeczematization.

When eczema is severe and does not respond to other forms of treatment, immunosuppressant drugs such as cyclosporin are sometimes prescribed. These dampen the immune system and can improve the eczema, but can cause side effects.

For severe itching, sedative antihistamines can be used but may cause drowsiness.

Atopic dermatitis is frequently associated with other atopic diseases like asthma, hay fever, and conjunctivitis. It is a chronic disease that often runs in the family, with symptoms that flair up or disappear temporarily. Atopic dermatitis afflicts humans, particularly young children, but is also a well characterized disease in domestic dogs.

Scar tissue is a mark left on damaged tissue both internally and externally such as on skin after it has healed after surgery or wounding. Scar tissue is dense fibrous connective tissue that forms over and/or around a healed wound or cut, and can affect negatively the elasticity of the skin and cause esthetic problems when visible on the skin and inconvenience. Extensive scar tissue can have negative effects on appearance and quality of life of individuals recovering from traumatic experiences such as burns.

Growth factors are key players in regulating proliferation and differentiation of cells and are involved in restructuring the epidermis and basal lamina upon injury or damage. They are important for the renewal of cells and thus, can counteract several aspects of aging and normalize keratinocyte differentiation, fibroblast growth and induce turnover and renewal of cells and cellular products.

Ito et al. (2009) teach that the expression of Matrix metalloproteinases (MMPs) and tissue inhibitors of metalloproteinase (TIMPs) in airway smooth muscle (ASM) cells could be involved in collagen turnover and migration of these cells and thus may contribute to airway remodeling. PDGF strongly up-regulates the expression of Matrix Metalloprotease-1 (MMP-1) at mRNA and protein levels. PDGF, when combined with TGF-beta, caused synergistic up-regulation of MMP-3. TIMP-1 was additively up-regulated by TGF-beta and PDGF.

Nakatani et al. (2009) teach that hyaluronan affects expression and protein levels of MMP-1 in periodontal ligament cells. Hyaluronan oligo (HAoligo) remarkably enhanced MMP-1 expression in both mRNA and protein levels, but no effect was shown on the expression of TIMP-1 and TIMP-2 mRNAs. It was suggested that HAoligo induces MMP-1 expression in HPDL cells, and p38MAPK plays a crucial role in signal transduction for MMP-1 inducted by HAoligo.

Although the examples of Ito and Nakatani are limited to signal pathways in airway smooth muscle and ligament cells they describe separately how growth factors and hyaluronan may affect the restructuring and turnover of components of extracellular matrix and basal lamina.

It is recognized that growth factors can have beneficial effects on various skin disorders and skin injuries and counteract effects of aging that are the result of impaired or deteriorating protective mechanisms at cellular level. Growth factors can promote cellular renewal and proliferation and are a natural component of the healing process of wounds.

The Epidermal Growth Factor (EGF) promotes the division of various epithelial cells originated in the ectoderm and mesoderm. It is extensively distributed in body fluid, especially in urine and breast milk (Carpenter, G. and Cohen, S., “Epidermal growth factor,” Ann Rev. Biochem., 48, 192-216 (1979)). It is a single polypeptide consisting of 53 amino acid residues and has a molecular weight of 6,200 Daltons (Campion, S. R. and Niyogi, S. K., “Interaction of epidermal growth factor with its receptor”). In 1962, Cohen isolated EGF from the gland beneath the chin of the mature male mouse. In 1972, Savage and Taylor identified the primary structure of mouse EGF and the location of three intramolecular disulfide bonds in EGF that are essential for physiological function.

EGF is believed to have an excellent effect on skin injuries because it strongly promotes the proliferation of epithelial cells, endothelial cells and fibroblasts, and also the migration and proliferation of epithelial cells to where they are deficient. Growth factors are key players in maintenance of tissue integrity and in cell to cell communication, thus playing a protective role in fighting degeneration of epidermal tissue.

U.S. Pat. No. 5,618,544 incorporated herein by reference in its entirety, discloses a cosmetic composition comprising EGF, TGF-a and FGF for decreasing cutaneous senescence and improving the appearance of skin.

U.S. Pat. No. 6,589,540 teaches that EGF remarkably enhances the effect of retinol used in cosmetics, and also effectively alleviates the skin irritation of retinol.

Growth factors such as PDGF are released at wound site during coagulation phase, and act as chemo-attractants for neutrophils, macrophages and fibroblasts. These cells play an important role in killing bacteria and removal of necrotic debris at the wound site. Activated macrophages release in turn growth factors that promote angiogenesis and communicate with the B-cell and T-cell mediated immune responses. Macrophages secrete TGF-beta, that stimulates fibroblasts to produce new extracellular matrix, and VEGFs that stimulate angiogenesis. Epithelization proceeds as keratinocytes divide and cover the wound bed. Thus, it is well established that growth factors are important mediators of healing process and studies indicate that G-CSF may be beneficial for treating infected diabetic ulcerations. EGF stimulates the proliferation of fibroblasts and keratinocytes.

FGF has proliferative effects on epithelial cells and has been observed to accelerate bone and wound healing in animal models. KGF -2 accelerates wound healing significantly, especially the closing of wounds.

As many other bioactive proteins, growth factors are sensitive to degradation when outside their natural environment, and it remains a challenge to formulate compositions with a long shelf life, preferably without need of refrigeration, to provide successful topical formulation products with recombinant proteins for the cosmetic and medical market. New and stable formulations will be highly appreciated, retaining long-term activity of the active proteins, and preferably without use of conventional preservatives.

### SUMMARY OF THE INVENTION

Applicants have previously developed successful methods to develop heterologous growth factors in transgenic plants. In the testing of the obtained products and development of compositions, applicants have developed new methods of treatment, based in hitherto unknown activity of growth factors for clinical dermatological use, administered topically in suitable formulation. Such clinical use is not limited to plant-produced growth factors, as those that are described and disclosed in applicants prior applications (see WO 2010/001417 and WO 2011/083500, which are hereby incorporated herein in full by reference). The present application is directed to use of growth factors for new and useful medical indications, wherein the growth factors are produced in host organisms or host cells other than plants or plant cells.

It is an object of this invention to provide stable formulations and compositions, and methods of use of non-plant derived stabilized heterologous recombinant growth factor in a hypoallergenic formulation, enabling the topical use of growth factors in cosmetic and skin care products. The stability of the growth factor over extended time is crucial to its use as a topical treatment, as proteins are by nature sensitive to breakdown and catalysis.

It is an important feature of the present invention to present a formulation of non-plant derived heterologous growth factor in a stabilizing composition with minimal number of ingredients, to obtain a non-allergenic, non-irritating composition that allows for the application and use of growth factors, not only to healthy skin but also to sensitive and challenged skin due to pathological condition such as but not limited to various types of eczema, including but not limited to atopic eczema and seborrhoic eczema, and psoriasis.

It is an aspect of the invention to provide compositions with non-plant derived heterologous growth factor with positive mitigating effects on scar tissue formation during and after the healing of a wound, burn, pustule, ulcer, lesion or surgery, such as medical surgery and plastic surgery. This embodiment of the invention is particularly useful in reducing the signs of scar tissue on skin, thus improving the complexion and healing of ruptured skin, improving the appearance of the skin.

In an aspect of the present invention, stabilized growth factors formulated according to the invention can be used to improve success of hair transplants by treatment of excised follicle units (FU) during and after the transplantation surgery.

A suitable growth factor or combination of two or more growth factors for the invention may be selected from non-plant derived recombinant growth factors including but not limited to Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g),Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2,BMP-3, BMP-4, BMP-5, BMP-6, BMP-7, BMP-8a), Hepatocyte Growth Factor (HGF), Leptin, Noggin, and Thymosin beta 4. These growth factors may be used according to the invention in healing of inflicted, pathological and surgical wounds and reduction/prevention of scar tissue formation. A selection of growth factors may be used for an ex vivo treatment in an operation such as hair-transplant, e.g. by immersing the excised follicle units in a solution containing recombinant growth factors to improve viability of the excised follicle units and to speed up and progress the healing process following the transplantation.

A choice of growth factors such as thymosin beta 4 and noggin are examples of preferred non-plant derived heterologous growth factors for this use of the invention, they are found to disrupt a refractive stage of cells in hair follicules at post-transplantational stage, and induce hair growth. The present invention provides compositions and means to treat scalp and follicles and/or follicle units (FU) in refractive stage with safe human growth factors, in a hypoallergenic formula to revitalize hair growth and for healing from the effects of the transplantation surgery.

It is a further aspect of the invention to provide compositions with one or more non-plant derived heterologous growth factor such as any of those above-mentioned and hyaluronan. Together, the effects of growth factors and hyaluronan on the metabolism of the skin can result in a positive synergistic effect on skin composition, normalizing cell differentiation, invigorating cell division and lead to renewal of components of the basal lamina, resulting in rejuvenated skin, wound healing and alleviation of persistent skin conditions and reduce inflammation.

In a further aspect of the invention to provide compositions with one or more growth factor such as any of those above-mentioned to treat Winter eczema or plantar dermatosis (“Winter foot”). In a specific embodiment, a composition is provided for treating Winter eczema, where the composition contains non-plant derived heterologous Epidermal growth factor (EGF).

In yet a further aspect, the present invention provides one or more non-plant derived heterologous growth factor isolated from a suitable host organism or cell expressing the non-plant derived heterologous protein, such as bacterial, yeast, or animal cells, including insect cells. The growth factors may also be used in other applications known to a skilled person in the art.

Further, the present invention provides the above mentioned growth factors for use as a medicament for any of the above stated conditions and ailments.

### DETAILED DESCRIPTION OF THE INVENTION

In the present context the term “non-plant derived heterologous growth factor” refers to a growth factor, preferably but not limited to a human growth factor, produced and obtained from a suitable non-plant transgenic host organism or transgenic cell expressing the heterologous growth factor, such as but not limited to bacterial, yeast, or animal cells including insect cells, including both cell cultures and transgenic organisms.

The term “heterologous” has the conventional meaning in the art, indicating that a heterologous protein is expressed by an organism/cell, which is different from the organism in which the same protein naturally is found.

The term human-like growth factor, that occurs in the present invention refers to the origin of the corresponding DNA sequence information. The DNA is a synthesized copy of the human DNA sequence for the growth factor in question.

Methods for introducing and expressing foreign genes in host cells or host organisms such as the above mentioned are well known in the art. A suitable host organism or host cell is selected and a suitable method for introduction of foreign gene selected. The term “transformation” or “genetic transformation” refers to the transfer of a nucleic acid molecule into the genome of a host organism or host cell, resulting in genetically stable inheritance. Host organisms containing the transformed nucleic acid fragments are referred to as “transgenic” organisms.

Growth factors that are suitably produced and used according to the present invention may be selected from but are not limited to the species and groups including Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2,BMP-3, BMP-4, BMP-5, BMP-6, BMP-7, BMP-8a), Hepatocyte Growth Factor (HGF), Leptin, Noggin, and Thymosin beta 4.

Hyaluronan is also called hyaluronic acid and hyaluronate, these terms are synonyms and interchangeable in the present context. Hyaluronan is an anionic, non-sulfated glycosaminoglycan distributed widely throughout connective, epithelial, and neural tissues.

The term skin care/dermatological composition as used herein encompasses both medical/pharmaceutical compositions for therapeutic dermatological applications as well as compositions for cosmetic use and compositions that can be used both for therapeutic and cosmetic use.

### Dosage

For topical therapeutic application in accordance with the invention, dose of growth factor is preferably in the range from 0.01 to 100 μg per gram of composition, and more preferably in the range 0.1 to 50 μg per gram. Local cosmetic compositions for the treatment of skin ageing or loss of hair preferably comprise from 0.2 to 50 μg of active substance per gram of composition.

The length of treatment varies depending on the pathology or on the desired effect. In the case of scleroderma treatment the application ranges from 1 day to 12 months according to the pathology severity. In the case of a treatment against natural or early ageing of the skin, the application ranges from 1 to 400 days, preferably for at least 30 days. Likewise, in the case of a treatment for preventing loss of hair or for promoting hair re-growth the application ranges from 1 to 400 days.

Dermatological compositions according to the invention can suitably be used for treatment of skin conditions including dry skin, eczema, dermatitis, rash, psoriasis, skin redness, and edema. Compositions of the invention are also useful for healing and reduction of scar tissue and healing and improving cracked skin on heels.

Numerous vehicles for topical application of cosmetic and pharmaceutical compositions are known in the art. See, e.g., Remington's Pharmaceutical Sciences, Gennaro, A. R., ed., 20th edition, 2000: Williams and Wilkins Pa., USA. All compositions usually employed for topically administering cosmetic compositions may be used, e.g., creams, lotions, gels, dressings, shampoos, tinctures, pastes, serums, ointments, salves, powders, liquid or semiliquid formulation, patches, liposomal preparations, solutions, suspensions, liposome suspensions, W/O or O/W emulsions, pomades and pastes and the like as long as the heterologous protein as active ingredient is stabilized. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used.

The compositions of the invention can be provided for parenteral, systemic or local use, comprising solutions, suspensions, liposome suspensions, W/O (water/oil) or O/W (oil/water) emulsions. In a preferred embodiment the active substance is formulated in a lyophilized form, mixed to suitable lyophilization additives and ready to be redissolved with therapeutically acceptable diluents. Useful lyophilization additives are: buffers, polysaccharides, sucrose, mannitol, inositol, polypeptides, amino acids and any other additive compatible with the active substance. In a preferred embodiment of the invention the active substance is dissolved in phosphate buffer (NaH₂PO₄/H₂O—Na₂HPO₄/2H₂O) in an amount such that the post-lyophilization growth factor/phosphate ratio is comprised between 1:1 and 1:2. Diluents suitable for parenteral use are: water, physiological solutions, sugar solutions, hydroalcoholic solutions, oily diluents, polyols, like glycerol, ethylene or polypropylene glycol, or any other diluent compatible with the administration method as for sterility, pH, ionic strength and viscosity.

Preferably, the vehicle of topical application is a formulation that is naturally anti-bacterial yet without any non-natural preservative or anti-microbial agent. It will be appreciated to use few ingredients and eliminate complex ingredients that may act as allergenics and/or irritants. The formulations should also ensure long term stability of the active protein ingredients, preferably providing long shelf life such as one year or longer at room temperature storage.

In a preferred embodiment the active compound, recombinant growth factor of choice, is added to a formulation suitable for topical application containing one or more of glycerol, a salt such as but not limited to sodium chloride, potassium chloride and calcium chloride, where calcium chloride is the most preferred, purified water, and ethanol, and preferably all of those. Such compositions are surprisingly shown to effectively stabilize the recombinant protein represented by the growth factor of choice. It is an aspect of the present invention that this formulation effectively stabilizes recombinant proteins whether or not the proteins are glycosylated. The formulation is preferably antibacterial by nature and therefore particularly suitable as a topical formulation for dermatological and cosmetic use. The composition of the invention may furthermore comprise an optional additive such as hyaluronic acid (hyalorunate).

In the case of emulsions or suspensions, the composition may contain suitable surfactants of non-ionic, zwitterionic, anionic or cathionic type commonly used in the formulation of medicaments. Oil/water (O/W) hydrophilic emulsions are preferable for parenteral systemic use, whereas water/oil (W/O) lipophilic emulsions are preferable for local or topic use.

Moreover, the compositions of the invention may contain optional additives like isotonic agents, such as sugars or polyalcohols, buffers, chelating agents, antioxidants, antibacterials.

Liquid forms according to the invention can comprise solutions or lotions. These may be aqueous, hydroalcoholic, like ethanol/water, or alcoholic and are obtained by solubilizing the lyophilized substance.

Alternatively, active substance solutions, may be formulated in form of gel by addition of known gelling agents, like: starch, glycerin, polyethylene or polypropylene glycol, poly(meth)acrylate, isopropyl alcohol, and hydroxystearate.

Other types of compositions for topic use are emulsions or suspensions in form of pomades, pastes, creams. W/O emulsions are preferable, providing a faster absorption. Examples of lipophilic excipients are: liquid paraffin, anhydrous lanolin, white vaseline, cetyl alcohol, stearyl alcohol, vegetable oils, mineral oils. Agents increasing cutaneous permeability, thereby facilitating the absorption, may advantageously be used. Examples of such agents are physiologically acceptable additives like polyvinyl alcohol, polyethylenglycol or dimethylsulfoxide (DMSO).

Other additives used in the topic compositions are isotonic agents, like sugars or polyalcohols, buffers, chelating agents, antioxidants, antibacterials, thickeners, dispersants.

It follows that the preparations may further contain conventional components usually employed in preparations described herein, including oils, fats, waxes, surfactants, humectants, thickening agents, antioxidants, viscosity stabilizers, chelating agents, buffers, preservatives, perfumes, dyestuffs, lower alkanols, and the like.

Delayed-release compositions for local or systemic use may be useful, and comprise polymers like polylactate, poly(meth)acrylate, polyvinylpyrrolidone, methylcellulose carboxymethylcellulose and other substances known in the art. Delayed-release compositions in form of subcutaneous implants based on, e.g. polylactate or other biodegradable polymers may be useful as well.

Though the active substance is preferably packaged in lyophilized and hence stable form, the pharmaceutical compositions advantageously comprise substances stabilizing the growth factor in the active form. Such stabilizers inhibit the formation of intermolecular disulfide bonds, thereby preventing the polymerization of the active substance. However, the amount of stabilizer should be carefully measured in order to concomitantly prevent the reduction of the active substance to the inactive monomeric form. Examples of such substances are: Cystein, Cysteamine, or glutathione in reduced form.

Non-limiting examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

### EXAMPLES

### Example 1

### Partially Purified Transgenic Cellular Extract Containing Growth Factor VEGF

Insect cell culture (Sf9) expressing human VEGF is processed by adding to the extract an IMAC chromatography resin that effectively binds the VEGF. The mixture of extract and resin is stirred in 50 mM potassium phosphate, 0.5 M NaCl, 50 mM imidazole; pH7.0 at +4° C. for 60 minutes. The IMAC resin is separated from the liquid by centrifugation at 5000×g for 15 minutes. The liquid phase is decanted off and the resin is resuspended in washing buffer (50 mM potassium phosphate, 0.5 M NaCl, 50 mM imidazole; pH7.0) and spun down and the liquid phase is decanted off the resin. The washing is repeated for 3 times. The resin is resuspended in elution buffer containing imidazole (50 mM potassium phosphate, 0.5 M NaCl, 500 mM imidazole; pH7.0) to elute the VEGF off the resin and after centrifugation the supernatant is decanted off the resin and run through gel filtration chromatography for buffer exchange. The resulting protein peak is analyzed on SDS-PAGE and Western blot.

### Example 2

### Stabilizing Formulations for Gowth Fctor

The following examples illustrate formulations of the cosmetic composition according to the present invention but are not intended to limit the invention in any way.

### Formulation 1: Skin Softener (Skin Lotion, Serum)

Stable compositions can be prepared buffered in the pH range of 6-9.

| Ingredients | Amounts (% by weight) |
|---|---|
| EGF (Epidermal Growth Factor) | 0.00025 |
| Glycerol | 10-90% |
| Calcium Chloride | 0.1 mM-200 mM |
| Purified water | q.s |
| Sodium hyaluronate | 0.01-2 |
| Ethanol | 0.1-10 |

### Formulation 2: Nutrient Emulsion (Milk Lotion)

| Ingredients | Amounts (% by weight) |
|---|---|
| EGF | 0.0002 |
| Propyleneglycol | 6.0 |
| Glycerin | 4.0 |
| Triethanolamine | 1.2 |
| Tocopherylacetate | 3.0 |
| Liquid paraffin | 5.0 |
| Squalene | 3.0 |
| Makadamia nut oil | 2.0 |
| Polysorbate 60 | 1.5 |
| Sorbitan sesquioleate | 1.0 |
| Carboxyvinylpolymer | 1.0 |
| Flavor | 0.2 |
| Methylparaben | 0.2 |
| Imidazolidinyl Urea | 0.2 |
| Purified water | q.s. |

### Formulation 3: Nutrient Cream

| Ingredients | Amounts (% by weight) |
|---|---|
| VEGF | 0.0005 |
| Vaseline | 7.0 |
| Liquid paraffin | 10.0 |
| Wax | 2.0 |
| Polysorbate 60 | 2.0 |
| Sorbitan sesquioleate | 2.5 |
| Squalene | 3.0 |
| Propyleneglycol | 6.0 |
| Glycerin | 4.0 |
| Triethanolamine | 0.5 |
| Carboxyvinylpolymer | 0.5 |
| Tocopherylacetate | 0.1 |
| Flavor | 0.2 |
| Methylparaben | 0.2 |
| Imidazolidinyl Urea | 0.2 |
| Purified water | q.s. |

### Formulation 4: Massage Cream

| Ingredients | Amounts (% by weight) |
|---|---|
| EGF | 0.0002 |
| Propyleneglycol | 6.0 |
| Glycerin | 4.0 |
| Triethanolamine | 0.5 |
| Wax | 2.0 |
| Tocopherylacetate | 0.1 |
| Polysorbate 60 | 3.0 |
| Sorbitan sesquioleate | 2.5 |
| Cetearyl alcohol | 2.0 |
| Liquid paraffin | 30.0 |
| Carboxyvinylpolymer | 0.5 |
| Flavor | 0.2 |
| Methylparaben | 0.2 |
| Imidazolidinyl Urea | 0.2 |
| Purified water | q.s. |

### Formulation 5: Facial Pack

| Ingredients | Amounts (% by weight) |
|---|---|
| TGF-a | 0.0005 |
| Propyleneglycol | 2.0 |
| Glycerin | 4.0 |
| Carboxyvinylpolymer | 0.3 |
| Ethanol | 7.0 |
| PEG-40 Hydrogenated Castor Oil | 0.8 |
| Triethanolamine | 0.3 |
| Flavor | 0.2 |
| Methylparaben | 0.2 |
| Imidazolidinyl Urea | 0.2 |
| Purified water | q.s. |

The formulations 1-5 can likewise be formulated with any alternative growth factor listed in the detailed description.

### Formulation 6: W/O Emulsion for Topic Application.

An amount of lyophilized substance comprising 20 μg active substance is brought to 5 ml 10% ethanol hydro-alcoholic solution comprising 10% DMSO. The solution is emulsified in sterilised vegetable oil for cutaneous application using a surfactant suitable for W/O emulsions having a <10 HLB coefficient. The emulsion contains active substance equal to about 2 μg/g of composition.

### Formulation 7: O/W Emulsion

An amount of lyophilized substance comprising about 20 μg active substance is solubilised in 5 ml of hydro-alcoholic solution comprising 30% DMSO and emulsified with a suitable surfactant in a vegetable oil-based lipophilic solvent. The resulting O/W emulsion contains the active substance at a concentration of about 3 μg/g composition.

### Formulation 8: Topical Composition in Form of Gel.

An amount of lyophilized substance comprising 100 μg of active substance is brought in 20 ml 10% ethanol hydro-alcoholic solution comprising 20% DMSO. Then, the solution is additioned with a mixture of polyethylene glycol (400-4000) and polypropylene glycol. The active substance is present in an amount equal to 2 μg/g composition. The gel is suitable for cosmetic application.

### Formulation 9: A Topical Gel Formulation Containing Carbomer (1%)

| | |
|---|---|
| EGF | 5 mg |
| Carbomer 934P | 1 g |
| Methyl paraoxybenzoate | 0.2 g |
| Propylene glycol | 20 g |
| Sodium hydroxide | q.s |
| Distilled water for injection | q.s |
| Total | 100 g |

The formulation is prepared by using the above-mentioned components in given amounts according to a conventional method. Specifically, methyl paraoxybenzoate is dissolved in appropriate amounts of distilled water for injection, Carbomer 934P is added to the solution and dispersed therein with sting. The pH of the solution is controlled with sodium hydroxide, the solution is blended with propylene glycol and sterilized by heating. Then, filtered and sterilized solution of EGF in distilled water for injection is added thereto to obtain 100 g of formulation.

### Formulation 10: A Topical Formulation Containing Poloxamer (15%)

| EGF | 2.5 | mg |
|---|---|---|
| Poloxamer 407 | 15 | g |
| Methyl paraoxybenzoate | 0.2 | g |
| Sodium hydrogen phosphate | 272.18 | mg |
| Sodium chloride | 666.22 | mg |
| Phosphoric acid | q.s | |
| Propylene glycol | 20 | g |
| Distilled water for injection | q.s. | |
| Total | 100 | g |

The formulation is prepared by using the above-mentioned components in given amounts according to a conventional method. Specifically, phosphate buffer is prepared by using sodium hydrogen phosphate, sodium chloride and phosphoric acid in given amounts. Methyl paraoxybenzoate as the preservative is dissolved to the phosphate buffer. Poloxamer 407(BASF, Germany) is added to the solution and dispersed therein with string. Then the solution is blended with propylene glycol, dispersed therein with stirring. Then, the pH of the solution is controlled with sodium hydroxide, the solution is blended with propylene glycol and sterilized by heating. Then, filtered and sterilized solution of EGF in distilled water for injection is added thereto to obtain 100 g of formulation.

### Formulation 11: A Cream Formulation Containing Carbomer (0.1%)

| | |
|---|---|
| EGF | 0.05 mg |
| Glycerin | 4.5 g |
| Methyl paraoxybenzoate | 0.15 g |
| Propyl paraoxybenzoate | 0.05 g |
| Carbomer 940 | 0.1 g |
| Steary alcohol | 1.75 g |
| Cetyl alcohol | 4.00 g |
| Span #60 | 0.50 g |
| Polyoxyl #40 stearate | 2.00 g |
| Triethanolamine | q.s |
| Distilled water for injection | q.s |
| Total | 100 g |

The formulation is prepared by using the above-mentioned components in given amounts according to a conventional method. Specifically, glycerin and methyl paraoxybenzoate are dissolved in appropriate amounts of distilled water for injection, Carbomer 940 (BF Goodrich, U.S.A.) is added to the solution and dispersed therein with stirring. Then, propyl paraoxybenzoate and the others are added to the solution and emulsified with melting. Then, the solution is sterilized after controlling pH with triethanolamine, and mixed with filtered and sterilized solution of EGF) in distilled water for injection to obtain 100 g of formulation.

### Example 3

### Antimicrobial Activity

The Example shows test of microbial challenge of a composition of the invention which is in accordance with Example 1, formulation 1, and the determination of antimicrobial preservation efficacy.

The composition was challenged with bacteria to establish antibacterial properties of the composition. 0.5 ml of liquid culture of Pseudomonas aeruginosa (strain ATCC 9027) was seeded onto 50 ml of stabilizing composition. Samples were incubated under standardized conditions and the number of bacteria determined based on methods described in detail for “Efficacy of Antimicrobial preservation” in European Pharmacopoeia 5.1.3.; Inoculum is prepared from stock culture of the bacteria, sterile suspension fluid containing 9 g/L of Sodium chloride. The culture is diluted with the fluid to obtain 107 bacteria per ml and 1 ml is added as inoculum in the container containing the stabilising composition and mixed thoroughly. The inoculated product is maintained at 22° C. protected from light. Samples of 1 ml are drawn from the inoculated product at specified time intervals and the number of bacteria determined by plate count.

| Time from seeding | Number (log10) of P. aeruginosa/ml of stabilizing |
|---|---|
| (days) | composition |
| 0 | 7.40 |
| 2 | 4.43 |
| 7 | 0.48 |
| 14 | 0 |
| 28 | 0 |

The results show clear antimicrobial activity of a composition of the present invention and fulfill the recommended efficacy of antibacterial activity according to European Pharmacopoeia for Topical preparations, without the use of conventional preservatives and antibacterial agents. Thus, these formulations are suitable for topical cosmetic and/or therapeutic compositions and circumvent possible side effects of potentially irritating preservatives, antimicrobial agents, and other additives which are compromising for sensitive skin.

### Example 4

### Treatment of Child Eczema

A subject, 6 month old girl who has suffered from children eczema in the face for 2 months. Steroids have been avoided and no methods or therapies had been effective against the eczema. Quality of life significantly affected as the constant scratching at the area by subject lead to unrest, crying and affected sleeping behavior.

After 2 days of application of 1-2 drops twice a day of the composition of the present invention, the affected area looked healthy and subject seized scratching and became more content and peaceful.

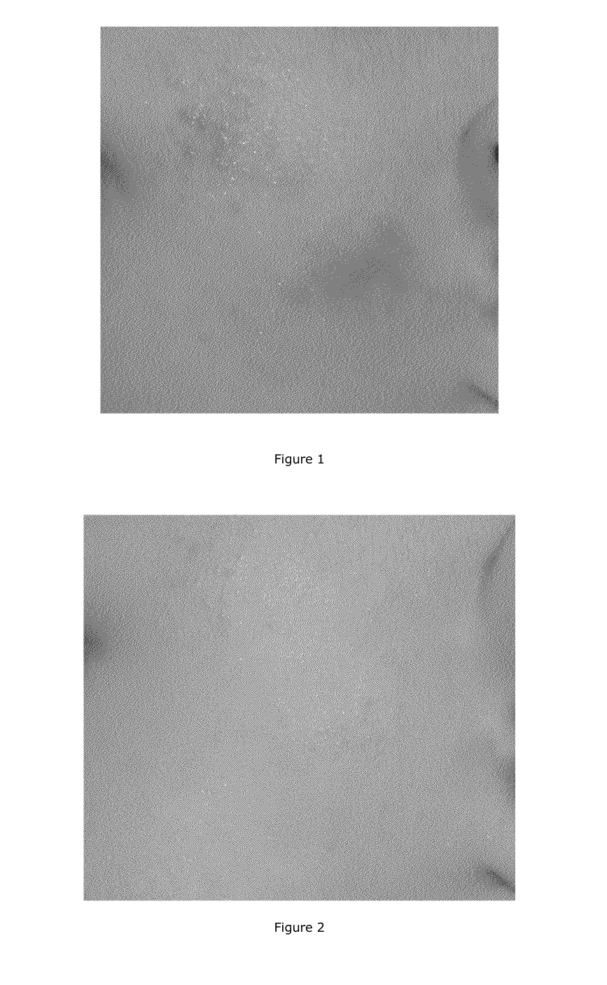

FIG. 1 shows eczema rash on left side of face of subject, area between the eye and ear, prior to treatment.

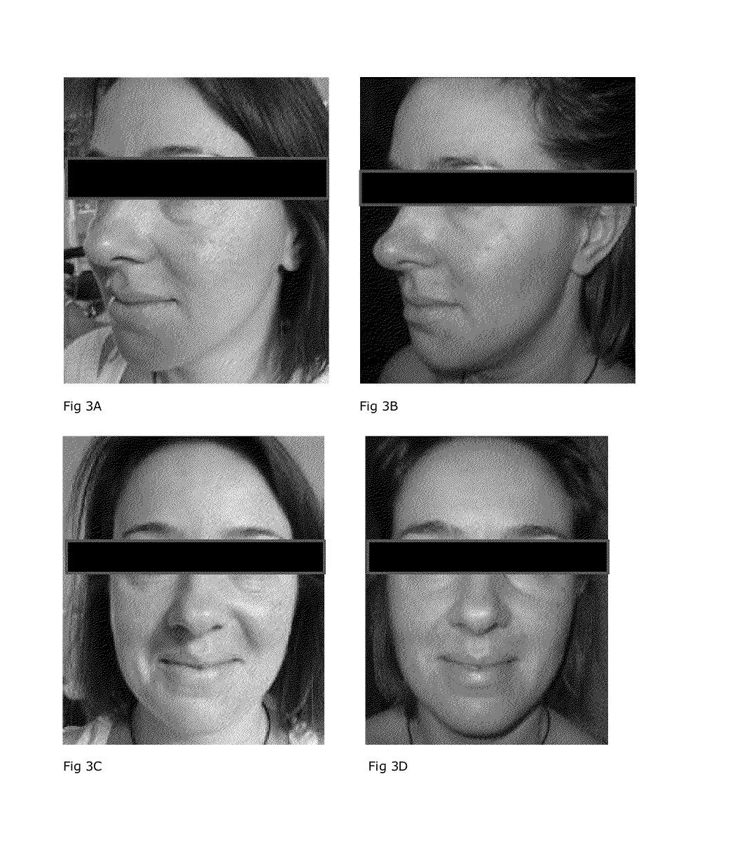

FIG. 2 shows same area after two days of treatment. 1-2 drops of the composition of the invention twice a day resulted in the disappearance of the eczema and seizing of the itchiness.

### Example 5

### Treatment of Child Eczema II

Atopic dermatitis is a type of eczema. It is an inflammatory, chronically relapsing, non-contagious and itchy skin disorder. A subject, 32 year old female diagnosed with atopic eczema in the facial area was treated with the human-like growth factor (EGF) containing composition of the present invention, as in Example 2, composition formulation 1, applying 2-4 drops of the composition twice a day. The condition of the skin was monitored by a medical doctor and documented by photography (FIGS. 3 A-D), before treatment (FIG. 3A and C) and after 4 weeks treatment (FIG. 3 B and D).

After 4 weeks of treatment redness, itchiness and inflammation had subsided and significant improvement in the condition of the skin was observed.

### Example 6

### Treatment of Psoriasis

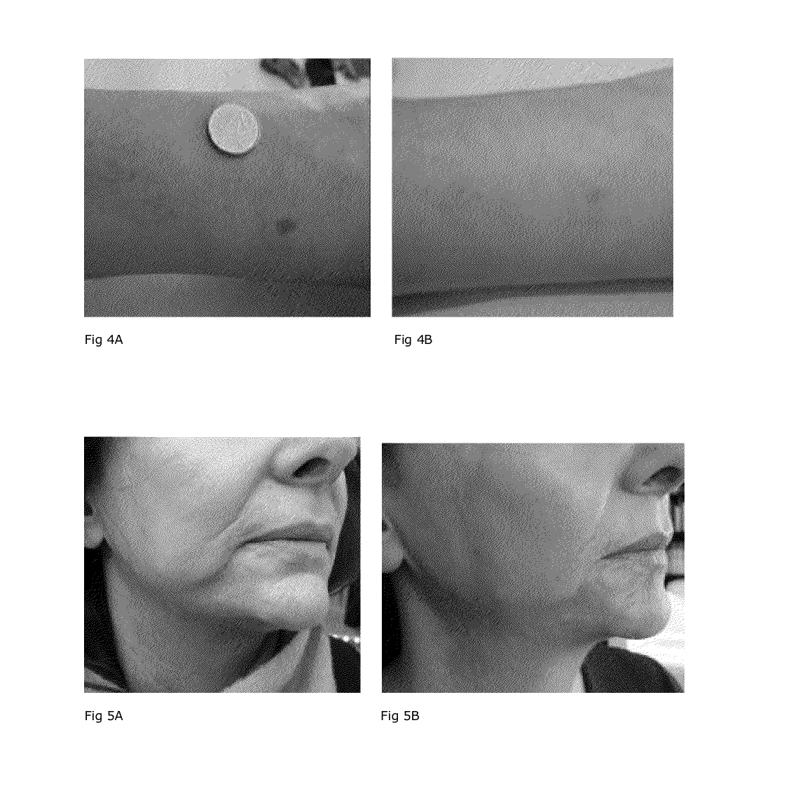

The most common form of psoriasis; psoriasis vulgaris or plaque psoriasis, is commonly seen as red and white hues of scaly patches appearing on the top first layer of the epidermis (skin). A 54 year old subject diagnosed with Psoriasis vulgaris on leg was treated with the human-like growth factor (EGF) containing composition of the present invention, as in Example 2, composition formulation 1 without CaCl2. A drop of the composition was rubbed onto the psoriasis plaque twice a day. The condition of the skin was monitored by a medical doctor and documented by photography before treatment (FIG. 4A) and after 4 weeks treatment (FIG. 4 B). After 4 weeks the psoriasis plaque had almost vanished.

### Example 7

### Relieving the Symptoms of Scleroderma

Scleroderma is a progressive autoimmune disorder that can disable its victims. In people with scleroderma, the body's immune system is activated, which results in inflammation and overproduction of thick layers of collagen. This resulting scar tissue can form anywhere in the body, especially in the skin. The cause of scleroderma is unknown. It affects women four times more frequently than men, and its symptoms usually occur in people between the ages of 35 and 65. A female subject of 54 years, diagnosed with Sclerodermia circumskripta in the skin was treated by a medical doctor with the human-like growth factor (EGF) containing composition of the present invention, as in Example 2, composition formulation 1 without ethanol and CaCl2. FIGS. 5 A and B show documentation of the treatment by physician before and after the treatment. The treatment resulted in less inflammation and smoother skin with smoother scars after 6 weeks of treatment of the facial area.

### REFERENCES:

Nakatani Y, Tanimoto K, Tanaka N, Tanne Y, Kamiya T, Kunimatsu R, Tanaka E, Tanne K. “Effects of hyaluronan oligosaccharide on the expression of MMP-1 in periodontal ligament cells.” Arch Oral Biol. 2009 Aug;54(8):757-63. Epub 2009 Jun. 11.
Technical specification sheet, EGF Recombinant Human Epidermal Growth Factor, Cell Sciences, MA, USA (http://www.cellsciences.com/PDF/CRE100.pdf) 5.1.3 Efficacy of Antimicrobial Preservation,: 528-529, European Pharmacopoeia 6.0

1. A skin care/dermatological composition comprising at least one non-plant derived heterologous growth factor and at least one pharmaceutically and/or cosmetically acceptable excipient.

2. The skin care/dermatological composition of claim 1 comprising an excipient formula that stabilizes the non-plant derived heterologous growth factor protein.

3. The skin care/dermatological composition of claim 1 comprising hyaluronic acid.

4. The skin care/dermatological composition of claim 3 wherein the hyaluronic acid is provided in a concentration in the range of about 0.01 to about 2% by weight.

5. The skin care/dermatological composition of claim 1, suitable for treatment of a skin condition selected from one or more of dry skin, eczema, dermatitis, cracked skin, rash, skin redness, scar tissue, psoriasis, and edema.

6. The skin care/dermatological composition of claim 2, wherein said protein stabilising stabilizing formula comprises glycerol, water, and calcium chloride.

7. The skin care/dermatological composition of claim 6, wherein said protein stabilizing formula comprises the following ingredients (% by weight): glycerol in the range from 10 to 90%, calcium chloride in the range from 0.1 mM to 200 mM, buffered in the pH range of 6-9 and purified water q.s.

8. The skin care/dermatological composition of claim 6, which does not contain a further antimicrobial agent or preservative.

9. The skin care/dermatological composition of claim 1, comprising more than one non-plant derived heterologous growth factor.

10. The skin care/dermatological composition of claim 1, wherein the at least one non-plant derived heterologous growth factor or more than one growth factors is isolated and purified to a level of purity in the range of about 70% to about 99.9%.

11. The skin care/dermatological composition of claim 1, wherein the at least one non-plant derived heterologous growth factor or more than one growth factor is selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g),Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2,BMP-3, BMP-4, BMP-5, BMP-6, BMP-7, BMP-8a),_Hepatocyte Growth Factor (HGF), Leptin, Noggin, and Thymosin beta 4.

12. The skin care/dermatological composition of claim 1, wherein said growth factor or cytokine originates from corresponding human gene sequence.

13. The skin care/dermatological composition of claim 1, wherein said non-plant derived growth factor or cytokine originates from synthesized gene corresponding to human gene sequence for respective growth factor or cytokine.

14. The skin care/dermatological composition of claim 1, wherein the composition is in the form selected from creams, lotions, gels, dressings, shampoos, tinctures, pastes, ointments, salves, powders, liquid or semi-liquid formulations, serums, patches, liposomal preparations, solutions, suspensions, liposome suspensions, W/O or O/W emulsions, ointments, pomades and pastes and a skin softener cream, a facial pack, a massage cream, and a nutrient cream or a nutrient emulsion.

15. The skin care/dermatological composition of claim 1, wherein the one or more non-plant derived heterologous growth factor isolated from a suitable non-plant host organism expressing the heterologous growth factor, such as bacterial, yeast, and animal cells, including insect cells.

16. A skin care/dermatological composition comprising at least one non-plant derived heterologous growth factor and a protein stabilizing formula comprising glycerol, and purified water q.s.

17. The skin care/dermatological composition of claim 16, further comprising a salt selected from sodium chloride, potassium chloride and calcium chloride.

18. The skin care/dermatological composition of claim 16, further comprising hyaluronic acid.

19. The skin care/dermatological composition of claim 17, comprising a non-plant derived heterologous growth factor and a protein stabilising formula comprising at least the following ingredients (% by weight): glycerol in the range from 10 to 90%, calcium chloride in the range from 0.1 mM to 200 mM, buffering agent and purified water q.s.

20. The skin care/dermatological composition of claim 16, which does not contain a further antimicrobial agent or preservative.

21. The skin care/ dermatological composition of claim 16, wherein said non-plant derived heterologous growth factor is one or more selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g),Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2, BMP-3, BMP-4, BMP-5, BMP-6, BMP-7, BMP- 8a), Hepatocyte Growth Factor (HGF), Leptin, Noggin, and Thymosin beta 4.

22. A non-plant derived heterologous growth factor use as a medicament.

23. A heterologous growth factor of non-plant genetic origin produced in a non-plant host organism or host cell, for use as a medicament for treatment of a skin condition selected from one or more of dry skin, eczema, dermatitis, cracked skin, rash, scar tissue, psoriasis, skin redness, and edema.

24. The heterologous non-plant derived growth factor of claim 22, selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (IFN-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2,BMP-3, BMP-4, BMP-5, BMP-6, BMP-7, BMP-8a), Hepatocyte Growth Factor (HGF), Leptin, Noggin, and Thymosin beta 4.

25. The heterologous non-plant derived growth factor of claim 22, which is Epidermal Growth Factor.

26. The heterologous non-plant derived growth factor of claim 22, which is produced in a host organism or host cell selected from the group consisting of bacterial cells, yeast cells, animals, and animal cells, including insect cells.

27. A skin care/dermatological composition comprising at least one non-plant derived heterologous growth factor and at least one pharmaceutically and/or cosmetically acceptable excipient suitable for treatment of plantar dermatosis.

28. The skin care/dermatological composition of claim 26 wherein said non-plant derived heterologous growth factor is Epidermal Growth Factor.
